(19) United States
(12) Patent Application Publication
(10) Pub. No.: US 2009/0082610 A1
(43) Pub. Date: Mar. 26, 2009

(54) OSTEO OR TISSUE HEALING DEVICE, KIT AND METHOD OF USING THE SAME

(76) Inventors: David A. WOLF, Houston, TX (US); Robert G. DENNIS, Raleigh, NC (US); Donnie RUDD, Sugar Land, TX (US)

Correspondence Address: LADAS & PARRY LLP, 224 SOUTH MICHIGAN AVENUE, SUITE 1600, CHICAGO, IL, 60604, US

(21) Appl. No.: 11/859,939

(22) Filed: Sep. 24, 2007

(51) Int. Cl. A61N 2/00

(52) U.S. Cl. 600/9

(57) ABSTRACT

An osteo or tissue healing device, kit and method of using the device to promote healing of compromised bone or tissue in a living mammal is disclosed. The osteo or tissue healing device includes a magnetic field emitter and a controlling circuit. The magnetic field emitter of the device is electrically coupled to a controlling circuit in which the magnetic field emitter is configured to provide a time variant magnetic field when driven by an electric pulse train from the controlling circuit in such a manner that the time variant magnetic field results in a magnetic (B) field exhibiting a magnetic slew rate of at least about 10 kiloGauss/sec. The controlling circuit of the device is electrically coupled to the magnetic field emitter and is configured to be powered by a power source, in which the controlling circuit is configured to output the electric pulse train driving the magnetic field emitter. It is thought that the steep magnetic field pulses and the relatively long inactive periods play important roles in promoting bone or tissue healing processes. The kit includes the unassembled components of the device. The method of using the osteo or tissue healing device includes the step of applying a time variant magnetic field to bone or tissue to promote healing of the bone or tissue.

### FIELD OF THE INVENTION

The present invention relates to medical device accessories, more particularly to an osteo or tissue healing device, kit and method of using the device for use in promoting healing of compromised bone or tissue in a living mammal is disclosed.

### BACKGROUND

This invention relates to a new and novel device, kit and method of using the device to improve the regeneration of bone and tissue in animals including humans, companion animals (such as dogs, cats, rabbits and the like) farm and working animals (such as horses, cows and the like) and laboratory and other animals, by the induction of a sequence of electromagnetic pulses with a mandatory relaxation period between the pulses. The tissue may include skeletal tissue, bone of all types, cartilage of all types, ligaments, and tendons. The period of time during which the electromagnetic energy is induced, or present, in the area of interest, usually an area in need of regeneration and growth is herein referred to as the “active” period. The period of time between the active periods is referred to as the “relaxation period” also called an “inactive period.” There may be a transition period between the active and relaxation periods usually required because the apparatus and real target system does not have an instantaneous response nor is it always advisable to induce such rapid response into the target area. The preferred embodiment utilizes an extremely short active period, 200 microseconds, during which an electromagnetic field is induced over the area of a broken bone or other tissue to be regenerated. The relaxation period is preferably 100 milliseconds. The relaxation period thus can occupy 99.8% of the time. It is thought that this long relaxation period is important because during this time the tissues, surrounding interstitial components, fluids, soluble ions, and other species are able to function in a normal manner not under the influence of the electromagnetic field or pulse. However, it is also thought that the intermittent temporary presence of an electromagnetic pulse momentarily disrupts the normal tissue behavior by freeing individual species to become again mobile, by increasing their chemical activity or availability, by synchronizing their diffusive or oscillatory behavior, by changing the conformation, presentation, size or other feature of active sites, by biasing the decay rate or path or outcome of unstable species, by transient entrainment or synchronization of molecular or ionic processes, by “pinging” or “ringing” the system allowing the system to restabilize in a different preferred rest state (analogous to shaking a sieve or tapping or vibrating a container to cause the contents to settle in a different arrangement, but on a much smaller scale so the physics is different but analogous, or other transient perturbation heretofore undescribed mechanistically but resulting in observable chemical and biological effects. Collectively we refer to these mechanisms as “activation” of the species in question. These Pulse-relaxation events presumably act upon molecules and charged species directly, and the time scale of the change in the electromagnetic field is such that it corresponds to the time constants for molecular events such as ion diffusion across membranes, ligand binding and release events, altering molecule associations, and protein folding (nano-seconds to micro-seconds).

Other schemes or processes, such as bulk mechanical shaking or sinusoidal electromagnetic perturbations of low frequency do not yield similar therapeutic effects due to the physical scale of the presumed mechanisms involved, which operate at very low Reynolds number. The amplitude of the field is limited so as to facilitate these extremely rapid electromagnetic transient perturbations, but the amplitude of the field is not thought to functionally or permanently disrupt the normal activity of the species in question. It is thought that the relaxation period is a key feature of this therapeutic approach that allows the species to resume normal unbiased interactions subsequent to the imposition of the transient magnetic field pulses but provides the benefit of the new arrangement of the component species. A possible key to understanding the therapeutic beneficial effects of compromised bones is that it can be thought of as being associated with the known early mobilization processes of bone or tissue healing, repair and maintenance. It appears that even externally imposed magnetic field pulse relaxation sequences can even provide a number of these therapeutic benefits to relative large scale limbs. Part of our scheme is envisioned to impose rapidly changing magnetic fields in and around compromised bones or tissue with a secondary electromagnetic state. It is also possible for the active and relaxation periods to consist of different electromagnetic waveforms over time, to be repetitive or non-repetitive, and to be regular or irregular in rhythm.

A similar and analogous situation may be effected by which the initial field introduced is electric, and the secondary induced field is magnetic. The ultimate outcome within the target area is the same. In this case the field generation means is by parallel plates or other means for generating the electric field whose time varying nature then induces the secondary magnetic field. The same concept of active and relaxation periods, and the description above, holds. The electromagnetic field pulses, active, and relaxation periods may also be generated and transmitted by an antennae arrangement.

Depending on the specific conditions under which the magnetic field pulse relaxation sequences are applied and the concomitant various shapes of the electromagnetic field waveforms that compose the sequences during these periods and the transition periods, different tissue function regulating effects may also be affected. In the preferred embodiment, improved bone healing from damage is the tissue function regulation achieved. The invention is particularly useful in regeneration of bone in humans and bone in companion animals (such as dogs, cats, rabbits and the like) farm and working animals (such as horses, cows and the like) and laboratory animals. Other background conditions and therapeutic interventions are envisioned to lead to their own particular regulatory outcome but the fundamental process of activation of the species by imposing these rapidly changing alternating magnetic field pulses coupled to relatively long relaxation periods for bio-molecular processes to proceed unimpeded is a constant theme in this invention. For example, even stem cells are thought to be affected by imposing these rapidly changing alternating magnetic field pulses coupled to relatively long relaxation periods which we believe enables an enhanced interaction of the multiple species by intermittent mobilization (during the active period) and, perhaps more importantly, the provision of the relaxation period during which assimilation and organization by natural bio-molecular processes may proceed. The particular target tissue effect, in character and degree, and particular regulation function may be determined and optimized by setting the various adjustable parameters such as duration of the active and relaxation periods and specific waveform thereof. In fact, the relaxation period may be adjusted to be of lesser duration than the active period, as tissue response demands, but is always associated with an active period and/or transition period(s). Other uses envisioned for this invention include delivery of chemotherapeutic agents into a tumor and may be termed as “bioavailability” enhancement by electromagnetic pulse relaxation techniques as a general class of applications. This class of electromagnetic pulse relaxation applications may be used in combination with and enhance other treatment modalities.

### SUMMARY OF THE INVENTION

The present device, kit and method of using, according to the principles of the present invention, overcomes a number of the shortcomings of the prior art by providing a novel osteo or tissue healing device, kit and method for use in promoting healing of compromised bone or tissue in a living mammal. In a preferred embodiment, the device includes a controlling circuit coupled to magnetic field emitter that emits relatively steep and sometimes short-lived magnetic field pulses during an active period. The osteo or tissue healing device may optionally provide an inactive phase in which no magnetic field pulses is imposed. Various alternate electromagnetic states, still distinct from the active period state, may permit relaxation to occur or enhance the relaxation period effects. It is thought that both these steep short-lived magnetic field pulses and the inactive periods play important roles in promoting healing processes in compromised bone and tissue. The kit includes the unassembled components of the device. The method of using the osteo or tissue healing device includes the step of applying a time variant magnetic field through the bone or tissue to promote healing of the bone or tissue.

To attain this, the present invention essentially comprises a controlling circuit coupled to magnetic field emitter that emits relatively steep magnetic field pulses during these active periods in which the osteo or tissue healing device also provides an inactive phase in which no magnetic field pulses are imposed. These steep magnetic field pulses may be rising magnetic field edges, or falling magnetic edges or both.

There has thus been outlined, rather broadly, the more important features of the invention in order that the detailed description thereof that follows may be better understood, and in order that the present contribution of the art may be better appreciated.

The invention may also include an optional power supply.

Numerous objects, features and advantages of the present invention will be readily apparent to those of ordinary skill in the art upon reading of the following detailed description of presently preferred, but nonetheless illustrative, embodiments of the present invention when taken in conjunction with the accompany drawings. In this respect, before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

It is therefore an aspect of the present invention to provide a new and improved osteo or tissue healing device that has many of the advantages of the prior osteo or tissue healing devices and minimizing a number of their disadvantages.

It is another aspect of the present invention to provide a new and improved osteo or tissue healing device that may be easily and efficiently manufactured and marketed.

An even further aspect of the present invention is to provide a new and improved osteo or tissue healing device that has a low cost of manufacture with regard to both materials and labor, and which accordingly is then susceptible of low prices of sale to the consuming public, thereby making the osteo or tissue healing device economically available to the buying public.

Still another aspect of the present invention is to provide an osteo or tissue healing device that provides in the apparatuses and methods of the prior art some of the advantages thereof, while simultaneously overcoming some of the disadvantages normally associated therewith.

Still another aspect of the present invention is to provide a kit comprising the un-interconnected elements of the osteo or tissue healing device. Including features for portability, mobility, low power consumption, durability, and maintainability.

Lastly, it is an aspect of the present invention to provide a new and improved method of using the kit comprising the steps of applying a time variant magnetic field through the bone or tissue to promote healing of the bone or tissue.

Unless otherwise defined, all scientific and technical terms used herein are to be construed as having the same meaning as commonly understood by one of ordinary skill in the art to which this invention pertains. Although methods and materials similar or equivalent to those described herein can be used in the practice or testing of the present invention, the preferred methods and materials are described below. All publications, patent applications, patents, and other references mentioned herein are incorporated by reference in their entirety. In case of conflict, the present document, including definitions, will control. Unless otherwise indicated, materials, methods, and examples described herein are illustrative only and not intended to be limiting.

There has thus been outlined, rather broadly, the more important features of the invention in order that the detailed description thereof that follows may be better understood, and in order that the present contribution of the art may be better appreciated.

Numerous other features and advantages of the present invention will be readily apparent to those of ordinary skill in the art upon reading of the following detailed description of presently preferred, but nonetheless illustrative, embodiments of the present invention when taken in conjunction with the accompany drawings. In this respect, before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

These together with other objects of the invention, along with the various features of novelty that characterize the invention, are pointed out with particularity in the claims annexed to and forming a part of this disclosure. For a better understanding of the invention, its operating advantages and the specific objects attained by its uses, reference should be had to the accompanying drawings and description matter in which there are illustrated embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 depicts a schematic view of an embodiment of the osteo or tissue healing device constructed in accordance with the principles of the present invention;

FIG. 2 depicts a perspective view of an embodiment of the osteo or tissue healing device;

FIGS. 3A, 3B, 3C, 3D and 3E depict a number of different embodiment configurations of the osteo or tissue healing device;

FIGS. 4A, 4B, 4C, 4D, 4E, and 4F depict a number of different electronic schemes of how the osteo or tissue healing device can be configured;

FIGS. 5A, 5B, and 5C depict a number of electromagnetic physical characteristics experienced by the magnetic field emitter during active and inactive periods;

FIGS. 6A, 6B, 6C, 6D, 6E, 6F, and 6G depict a number of embodiments of showing different magnetic field output patterns as a function of time;

FIGS. 7A, 7B, 7C, 7D, 7E, and 7F depict various ways the osteo or tissue healing device can be externally mounted to promote healing of a compromised bone of a living mammal;

FIG. 8 depicts the osteo or tissue healing device internally mounted to promote healing of a compromised bone;

FIG. 9 depicts an osteo or tissue healing device mounted onto a mammal;

FIG. 10 is an X-ray of the leg of a test rabbit immediately post surgery showing the piece of the radial bone removed with the ulna bone intact;

FIG. 11 shows the bone healing of a test rabbit with a natural non-invention healing;

FIG. 12 shows that the non-treatment rabbit again had no discernable healing of the bone after 4 weeks;

FIG. 13 shows the bone healing of a test rabbit having the benefit of this invention after 4 weeks;

FIG. 14 is a cross section of the surgical area of FIG. 13 showing that the bone has healed in a proper manner and that it is regenerated bone; and

FIG. 15 is a schematic of an exemplary electronic circuit utilized to drive the time variant magnetic field generated by the coil magnetic field emitter.

The same reference numerals refer to the same parts throughout the various figures.

### DETAILED DESCRIPTION

The following detailed embodiments presented herein are for illustrative purposes. That is, these detailed embodiments are intended to be exemplary of the present invention for the purposes of providing and aiding a person skilled in the pertinent art to readily understand how to make and use of the present invention.

Accordingly, the detailed discussion herein of one or more embodiments is not intended, nor is to be construed, to limit the metes and bounds of the patent protection afforded the present invention, in which the scope of patent protection is intended to be defined by the claims and their equivalents thereof. Therefore, embodiments not specifically addressed herein, such as adaptations, variations, modifications, and equivalent arrangements, should be and are considered to be implicitly disclosed by the illustrative embodiments and claims described herein and therefore fall within the scope of the present invention.

Further, it should be understood that, although the claimed method may be shown and described as being in a sequence or temporal order, the steps of any such method are not limited to being carried out in any particular sequence or order, absent an indication otherwise. That is, the claimed method steps are to be considered to be capable of being carried out in any sequential combination or permutation order while still falling within the scope of the present invention.

Additionally, it is important to note that each term used herein refers to that which a person skilled in the relevant art would understand such term to mean based on the contextual use of such term herein. To the extent that the meaning of a term used herein, as understood by the person skilled in the relevant art based on the contextual use of such term, differs in any way from any particular dictionary definition of such term, it is intended that the meaning of the term as understood by the person skilled in the relevant art should prevail.

Furthermore, a person skilled in the art of reading claimed inventions should understand that “a” and “an” each generally denotes “at least one,” but does not exclude a plurality unless the contextual use dictates otherwise. And that the term “or” denotes “at least one of the items,” but does not exclude a plurality of items of the list.

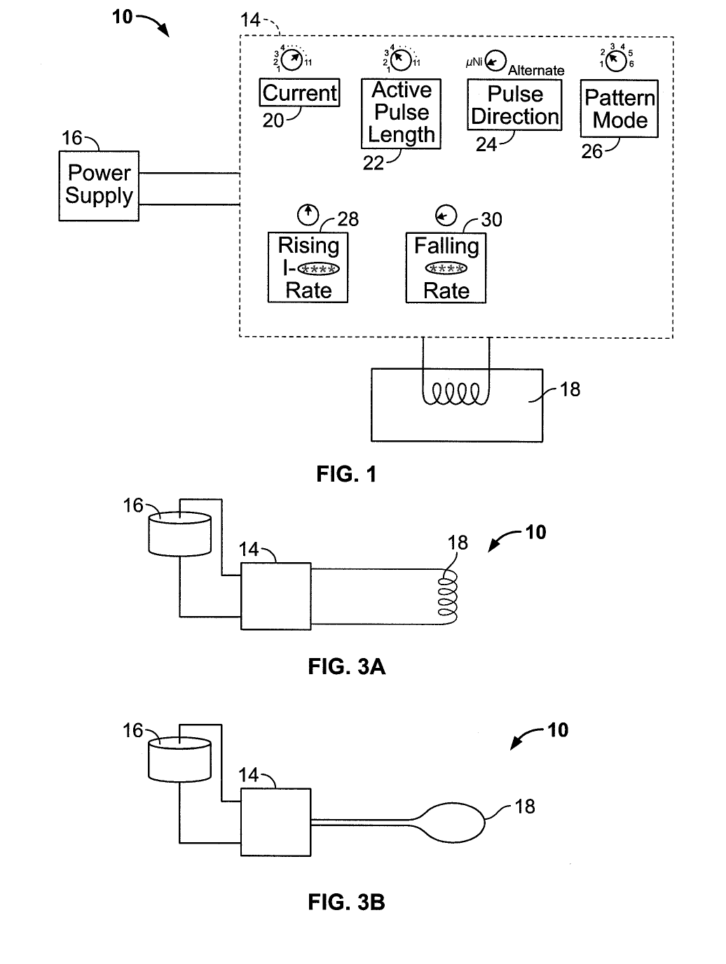

Referring now to the drawings, and in particular FIGS. 1 to 15 thereof, one embodiment of the present invention is shown and generally designated by the reference numeral 10. This embodiment of an osteo or tissue healing device 10 for promoting healing of a compromised bone 12 in a living mammal 34 comprises a controlling circuit 14 and a magnetic field emitter 18. The controlling circuit 14 is configured to be powered by a power source 16 and is configured to output an electric pulse train. The electric pulse train outputted from the controlling circuit 14 comprises an output current, an electrical cycle period, an electrical active and inactive period, a peak voltage amplitude, and a peak current amplitude. The magnetic field emitter 18 electrically coupled to the controlling circuit 14 is configured to provide a time variant magnetic field when driven by the electric pulse train of the controlling circuit 14. The magnetic field emitter 18 that is electrically coupled to the controlling circuit 14 is configured to provide a time variant magnetic field comprising a magnetic (B) field exhibiting a magnetic slew rate of at least about 10 kiloGauss/sec when driven by the electric pulse train from the controlling circuit 14. The time variant magnetic field can comprises a magnetic field, a magnetic cycle having an active and inactive duty, and peak magnetic amplitude. The magnetic field of the time variant magnetic field can be restricted to exhibit a magnetic slew rate (either rising or falling, or both rising and falling) of at least about 10 kiloGauss/sec. The magnetic field of the time variant magnetic field can be configured to exhibit a magnetic cycle period at least about 0.01 Hertz. The magnetic field of the time variant magnetic field can be restricted to exhibit a magnetic field active duty between about 0.01 to 50 (preferably 0.01 to 2) percent of the cycle period wherein the magnetic active field duty is defined as when the magnetic field emitter 18 emits the magnetic field. Accordingly, the magnetic field of the time variant magnetic field can be restricted to exhibit a magnetic inactive duty being between about 50 to 99.99 (preferably 98 to 99.99) percent of the cycle period in which the magnetic field inactive duty is defined as when the magnetic field emitter 18 does not emit the magnetic field.

The electric pulse train of the controlling circuit 14 may be any know form as long as the B field exhibits a magnetic slew rate of at least about 10 kilo Gauss/sec. Accordingly the electric pulse train of the controlling circuit 14 may exhibit an output current having a rising slew rate of at least about 1 Amperes/sec and has a failing slew rate of at least about 1 Amperes/sec; the electrical cycle period may be at least about 0.01 Hertz; the electrical active period may be between about 0.01 to 2 percent of the electrical cycle period; the electrical inactive period may be between 98 to 99.99 percent of the electrical cycle period; the peak voltage amplitude may be at least about +3 Volts; and the peak current amplitude may be at least about 1 Ampere.

The device 10 is subject to almost an infinite number of design variations as long as the device 10 can produce a magnetic slew rate (either rising or falling, or both rising and falling) of at least about 10 kiloGauss/sec. For instance, one variation is that the magnetic field of the time variant magnetic field can be restricted to exhibit a slew rate (either rising or falling, or both rising and falling) being between about 25 to about 1000 kiloGauss/sec. Another variation is that the magnetic field of the time variant magnetic field can be configured to exhibit a magnetic cycle period between about 0.01-1000 Hertz. Yet another variation is that the magnetic field of the time variant magnetic field can be restricted to exhibit a magnetic field active duty between about 0.01 to 50 (preferably 0.01 to 2) percent of the cycle period wherein the magnetic active field duty defined as when the magnetic field emitter 18 emits the magnetic field. Still yet another variation is that the magnetic field of the time variant magnetic field is restricted to exhibit a magnetic inactive duty being between about 50 to 99.99 (preferably 98 to 99.99) percent of the cycle period wherein the magnetic field inactive duty defined as when the magnetic field emitter 18 does not emit the magnetic field. Even yet another variation is that the magnetic field of the time variant magnetic field can be restricted to exhibit a peak magnetic amplitude being between about −20 to +20 Gauss. One variation in the design of the controlling circuit 14 is that it is configured to exhibit an electrical current slew rate (either rising or falling, or both rising and falling) between about 10 to about 1000 Amperes/sec. Another variation of the controlling circuit 14 is that the output current of the electric pulse train outputted from the controlling circuit 14 can be configured to exhibit a falling slew rate being between about 10 to about 1000 Amperes/sec. Yet another variation of the controlling circuit 14 is that it can be configured to the output the electric pulse train to exhibit an electrical cycle period being between about 0.01-100 Hertz. Still another variation of the controlling circuit 14 is that the output current of the electric pulse train outputted from the controlling circuit 14 can be restricted to exhibit an electrical active period between about 0.01 to 50 (preferably 0.01 to 2) percent of the electrical cycle period wherein the electrical active period defined as when the output current is outputted. Yet another variation of the controlling circuit 14 is that the output current of the electric pulse train outputted from the controlling circuit 14 can be restricted to exhibit an electrical inactive period between 50 to 99.99 (preferably 98 to 99.99) percent of the electrical cycle period wherein the electrical inactive period defined as when the output current is not outputted. Still yet another variation of the controlling circuit 14 is that the output current of the electric pulse train outputted from the controlling circuit 14 can be configured to exhibit a peak voltage amplitude being between about −5 to +5 Volts and to exhibit a peak current amplitude being between about −5 to +5 kiloAmps. Even yet another variation of the controlling circuit 14 is that the electric pulse train of the controlling circuit 14 is that the output current can be configured to exhibit a rising electrical current slew rate between about 10 to about 1000 Amperes/sec and to exhibit a falling electrical slew rate being between about 10 to about 1000 Amperes/sec. Still another variation of the controlling circuit 14 is that the electrical cycle period can be configured to be between about 0.01-100 Hertz. Yet another variation of the controlling circuit 14 is that the electrical active period can be configured to be between about 0.01 to 2 percent of the electrical cycle period and that the electrical inactive period can be configured to be between 98 to 99.99 percent of the electrical cycle period. Even yet another variation of the controlling circuit 14 is that the peak voltage amplitude can be configured to be between about 1 to 10 Volts. Still yet another variation of the controlling circuit 14 is that the peak current amplitude can be configured to be being between about 1 to 10 Amps.

The magnetic field emitter 18 of the osteo or tissue healing device 10 may be made of any known material selected from the group consisting of a coil magnetic field emitter 18, a plurality of coil magnetic field emitters 18, a plurality of loop magnetic field emitters 18, and an antenna magnetic field emitter. Further, the magnetic field emitter 18 may exhibit any known inductance value.

The controlling circuit 14 of the osteo or tissue healing device 10 may have an optional current switch 20 that may be added to the controlling circuit 14 of the osteo or tissue healing device 10 in which the optional current switch 20 is configured to control the output current of the electric pulse train outputted from the controlling circuit 14. The controlling circuit 14 of the osteo or tissue healing device 10 may have an optional cycle length switch 22 that may be added to the controlling circuit 14 of the osteo or tissue healing device 10 in which the optional cycle length switch 22 is configured to control the electrical cycle period of the electric pulse train outputted from the controlling circuit 14. The controlling circuit 14 of the osteo or tissue healing device 10 may even have an optional pulse direction switch 24 that may be added to the controlling circuit 14 of the osteo or tissue healing device 10 in which the optional pulse direction switch 24 is configured to control the peak voltage and current amplitudes of the electric pulse train outputted from the controlling circuit 14. The controlling circuit 14 of the osteo or tissue healing device 10 may also have an optional output mode switch 26 that may be added to the controlling circuit 14 of the osteo or tissue healing device 10 in which the output mode switch 26 is configured to control various patterns of the electric pulse train outputted from the controlling circuit 14. Alternating polarity, or other sequences with low net DC values over time may yield the advantage of not introducing or accumulating long term net electric or magnetic motive forces. In cases where such accumulated forces are desirable, the instrumentation may be adjusted to produce such, and in a degree found to optimize the tissue response. Still yet the controlling circuit 14 of the osteo or tissue healing device 10 may also have an optional rising slew rate switch 28 that may be added to the controlling circuit 14 of the osteo or tissue healing device 10 in which the rising slew rate switch 28 is configured to control the output current rising slew rate of the electric pulse train outputted from the controlling circuit 14. The controlling circuit 14 of the osteo or tissue healing device 10 may have an optional falling slew rate switch 30 that may be added to the controlling circuit 14 of the osteo or tissue healing device 10 in which the falling slew rate switch 30 configured to control the output current falling slew rate of the electric pulse train outputted from the controlling circuit 14. The “slew” or rate of change of the energizing signal may be constant or variable, and in practical terms, variability is accepted in most practically realizable implementations. Variability, such as “tapering” or “wave shaping” at inflection points and sharp signal transition points may be optionally introduced and occasionally may yield an advantage in healing of target tissue response.

The osteo or tissue healing device 10 may optionally comprise the power source 16 electrically coupled to the controlling circuit 14. The optional power source 16 may be selected from the group consisting of a battery power source 16, a high capacity capacitor power source 16, and an electrical outlet power source 16.

An embodiment of a kit for an osteo or tissue healing device 10 may comprise a a magnetic field emitter 18 coupleable to a controlling circuit 14. The magnetic field emitter 18 may be configured to be electrically coupled to the controlling circuit 14 in which the magnetic field emitter 18 is configured to provide a time variant magnetic field when driven by the electric pulse train of the controlling circuit 14. The time variant magnetic field comprises a magnetic (B) field exhibiting a magnetic slew rate of at least about 10 kiloGauss/sec. The controlling circuit 14 may be configured to be powered by a power source 16 and is also configured to output an electric pulse train.

The controlling circuit 14 of the kit of the osteo or tissue healing device 10 may optionally have a current switch 20 which is configured to control the electrical cycle period of the electric pulse train to output from the controlling circuit 14. The controlling circuit 14 of the kit of the osteo or tissue healing device 10 may also optionally have a cycle length switch 22 configured to control the electrical cycle period of the electric pulse train outputted from the controlling circuit 14. The controlling circuit 14 of the kit of the osteo or tissue healing device 10 may also optionally have a pulse direction switch 24 configured to control the peak voltage and current amplitudes of the electric pulse train outputted from the controlling circuit 14. The controlling circuit 14 of the kit of the osteo or tissue healing device 10 may also optionally have an output mode switch 26 configured to control various patterns of the electric pulse train outputted from the controlling circuit 14. The controlling circuit 14 of the kit of the osteo or tissue healing device 10 may also optionally have a rising slew rate switch 28 configured to control the output current rising slew rate of the electric pulse train outputted from the controlling circuit 14. The controlling circuit 14 of the kit of the osteo or tissue healing device 10 may also optionally have a falling slew rate switch 30 configured to control the output current falling slew rate of the electric pulse train outputted from the controlling circuit 14.

The magnetic field emitter 18 of the kit of the osteo or tissue healing device 10 may be any known commercially available magnetic field emitter 18. Some magnetic field emitters 18 may be selected from the group consisting of a coil magnetic field emitter 18, a plurality of coil magnetic field emitters 18, a plurality of loop magnetic field emitters 18, and an antenna magnetic field emitter 18.

An optional power source 16 may be added to the kit of the osteo or tissue healing device 10 in which the optional power source is configured to be electrically coupled to the controlling circuit 14. The power source 16 of the kit of the osteo or tissue healing device 10 may be any known power source 16 in which some preferred power sources 16 may be selected from the group consisting of a battery, a high capacity capacitor, and an electrical outlet.

An optional stabilizing agent 32 may be added to the kit of the osteo or tissue healing device 10 in which the stabilizing agent 32 may be any known and commercially available stabilizing agents 32. Some preferred stabilizing agents 32 include those selected from the group consisting of an external applied plaster cast stabilizing agent 32, an externally applied splint stabilizing agent 32, an external traction mounting stabilizing agent 32 and an internally applied shank stabilizing agent 32.

One method for promoting healing of a compromised bone 12 in a living mammal 34 comprises the step of applying a time variant magnetic field through the portion of the bone 12 to promote healing of the bone 12. The time variant magnetic field of the applying step comprises a magnetic field having a slew rate (either a rising or a falling, or both a rising and falling) of at least 10 kiloGauss/sec. The direction of the magnetic field may be adjusted to be longitudinal (with the bone), transverse (across the bone), or intermediate between these extremes. Furthermore, the field may not even have to be relatively homogeneous through space in character of adjustable scalar or directional parameters. In fact, due to non-ideal implementations, such inhomogeneities are generally expected and may be adjusted to obtain optimal tissue response, such as bone healing.

The applying step may last for any known length of time. One variation is that the applying step is applied for a duration of at least two weeks without interruption. Another variation of the applying step is that it lasts for a duration of at least two weeks and is performed at least 8 hours in each day on the mammal 34 during the duration of the applying step.

The time variant magnetic field may be applied in any known direction. One embodiment is that the time variant magnetic field is always applied along a substantially identical direction during the applying step, whereby the time variant magnetic field being a unidirectional time variant magnetic field. Another embodiment is that the time variant magnetic field is alternately applied along substantially alternate opposite directions during the applying step, whereby the time variant magnetic field being an alternating bidirectional time variant magnetic field.

Another embodiment is that the time variant magnetic field is applied using a current pulse train through a magnetic field emitter generated by a circuit. The time variant magnetic field may exhibit any known periodicity such as having an active duty between about 0.1 to 1 percent of the cycle period; the rising edge magnetic slew rate of at least about 10 kiloGauss/sec; and the falling edge magnetic slew rate being of at least about 10 kiloGauss/sec. One embodiment of the electric pulse train comprises an output current exhibiting a rising slew rate between of at least 1 Amperes/sec; the output current exhibiting a falling slew rate of at least about 1 Amperes/sec; an electrical cycle period being at least about 0.01 Hertz; an electrical active periodicity may be any function, such as being between about 0.01 to 2 percent of the electrical cycle period wherein the electrical active period defined as when the output current is outputted; an electrical inactive period between 98 to 99.99 percent of the electrical cycle period wherein the electrical inactive period defined as when the output current is not outputted; a peak voltage amplitude being between about −5 to +5 Volts; and a peak current amplitude being between about −5 to +5 kiloAmps.

It is envisioned that the present method is suitable for promoting healing of compromised bones 12 selected from the group consisting of a simple fracture compromised bone 12, a compound fracture compromised bone 12, a cracked compromised bone 12, a strained compromised bone 12, and a low density compromised bone 12.

It is envisioned that the present method is suitable for promoting healing of compromised bones in mammals 34 selected from the group consisting of a human, a domesticated dog, a domesticated cat, a rat, a mouse, a guinea pig, a rabbit, a horse, a cow, a llama, an alpaca, a mule, a donkey, a gorilla, a chimpanzee, a lemur, a rhinoceros, a monkey, a bat, a bison, a camel, a wolf, a coyote, a fox, a jackal, tiger, an oryx, a water buffalo, a elephant, a giraffe, an antelope, a deer, an elk, a lion, a cheetah, a panda, a leopard, a puma, a serval, an opossum, a kangaroo, a platypus, an armadillo, a lemur, a muskox, a baboon, a zebra, a pig, a koala, a tasmanian devil, a manatee, and a wombat. It expressly includes companion animals (such as dogs, cats, rabbits and the like) farm and working animals (such as horses, cows and the like) and laboratory and other animals.

An optional aligning step may be added to the method in which the aligning step is used to align the bone 12 in a desired orientation.

An optional stabilizing step may be added to the method in which the stabilizing step is used to stabilize the bone 12 with a stabilizing agent 32.

An optional mounting step may be added to the method in which the mounting step is used to mount a magnetic field emitter 18 near a portion of the bone 12. The mounting step of the magnetic field emitter 18 may be performed in any known manner such as being mounted external relative to the mammal 34, i.e., without surgery or being mounted internally relative to the mammal 34, e.g., using surgical techniques to mount the magnetic field emitter. The magnetic field emitter 18 may be any known magnetic field emitter. Some preferred embodiments of magnetic field emitters 18 are selected from the group consisting of a coil magnetic field emitter 18, a plurality of coil magnetic field emitters 18, and a plurality of loop magnetic field emitters 18.

An optional turning off step may be added to the method in which the turning off step is used to turn off the time variant magnetic field after a substantial amount of healing of the bone 12 has occurred.

An optional withdrawing step may be added to the method in which the withdrawing step is used to withdraw the magnetic field emitter 18 away from the portion of the bone 12 subsequent to when the bone 12 being substantially healed. An optional stabilizing step may be added to the method in which the stabilizing step is used to stabilize a portion of the bone 12 subsequent to when the bone 12 being substantially healed. The stabilizing agent 32 may be any known bone stabilizing agent 32. Some preferred embodiments of stabilizing agents 32 are selected from the group consisting of an external applied plaster cast stabilizing agent 32, an externally applied splint stabilizing agent 32, an external traction mounting stabilizing agent 32 and an internally applied shank stabilizing agent 32.

Referring now to FIG. 1 which depicts a schematic view of an embodiment of the osteo or tissue healing device 10 showing the optional power supply 16 electrically coupled to the controlling circuit 14. The controlling circuit 14 is shown having the optional current switch 20, the optional cycle length switch 22, the optional pulse direction switch 24, the optional output mode switch 26, the optional rising slew rate switch 28, and the optional falling slew rate switch 30. Also shown is the magnetic field emitter 18 electrically coupled to the controlling circuit 14.

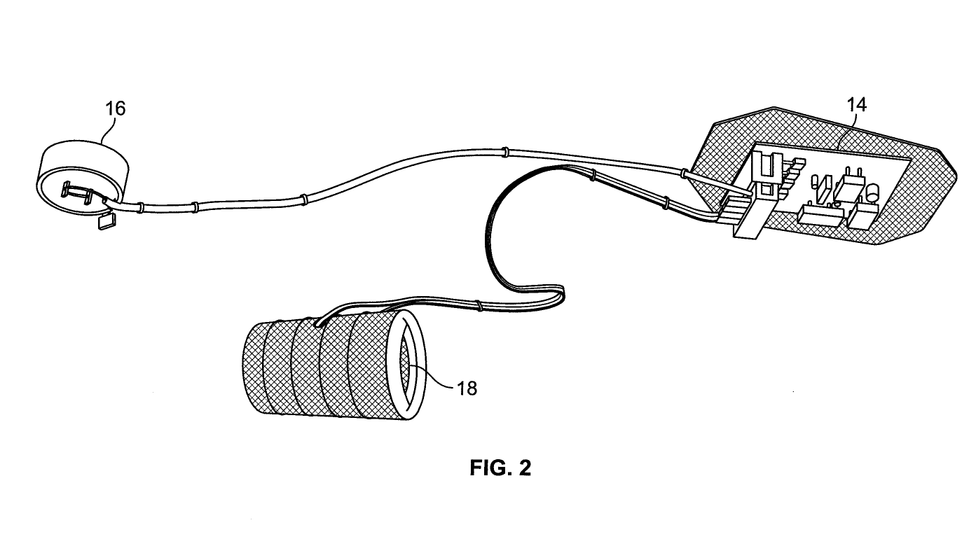

Referring now to FIG. 2 which depicts a perspective view of an embodiment of the osteo or tissue healing device 10 showing the optional power supply 16 and the magnetic field emitter 18 electrically coupled to the controlling circuit 14.

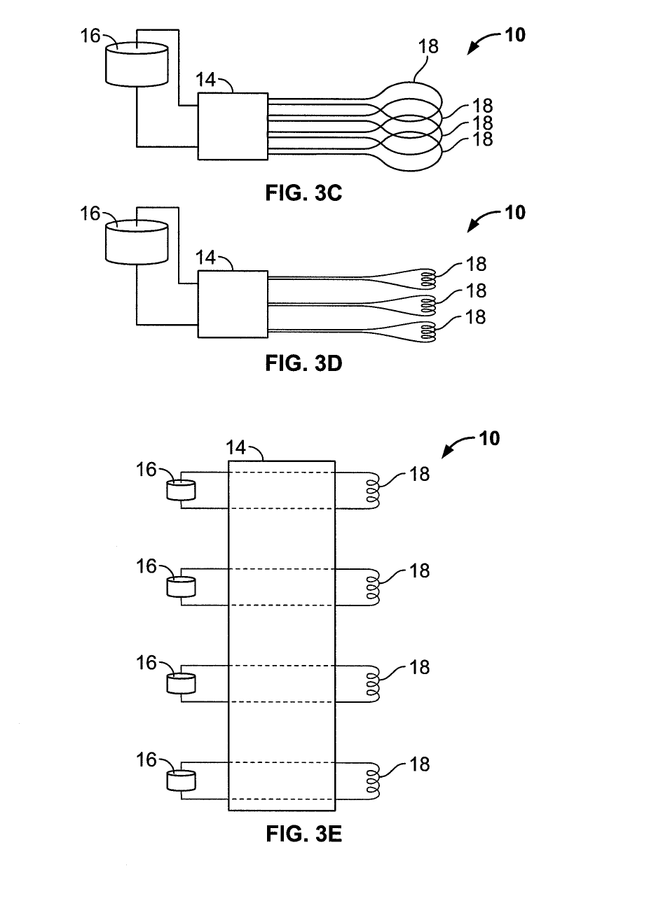

Referring now to FIGS. 3A, 3B, 3C, 3D and 3E that depict a number of different embodiments of the osteo or tissue healing device 10. The osteo or tissue healing device 10 is shown having any number of different designs or configurations. FIG. 3A illustrates one configuration of the controlling circuit 14 which is coupled to only one coil magnetic field emitter 18 and is powered by only one power supply 16. FIG. 3B illustrates another configuration of the controlling circuit 14 which is coupled to a plurality of loop magnetic field emitters 18 and is powered by only one power supply 16. FIG. 3C illustrates yet another configuration of the controlling circuit 14 that is coupled to a plurality of loop magnetic field emitters 18 and is powered by only one power supply 16. FIG. 3D illustrates still yet another configuration of the controlling circuit 14 that is coupled to a plurality of coil magnetic field emitters 18 and is coupled to a plurality of power supplies 16. In FIG. 3E each power supply 16 is shown configured via the controlling circuit 14 to individually drive only a single corresponding coil magnetic field emitters 18.

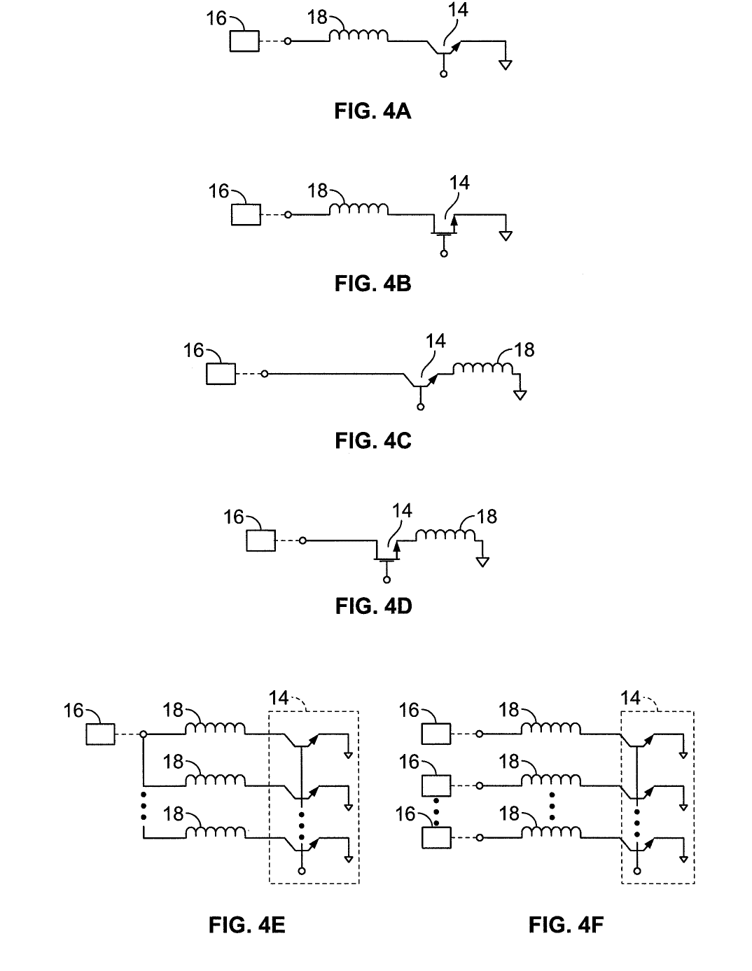

Referring now to FIGS. 4A, 4B, 4C, 4D, 4E, and 4F that depict a number of different electronic schemes of how the osteo or tissue healing device 10 can be configured. These electronic schemes are depicted to illustrate just a few of the infinite number of electronic configurations of the osteo or tissue healing device 10 as long as each can realize the invention as described in the claims.

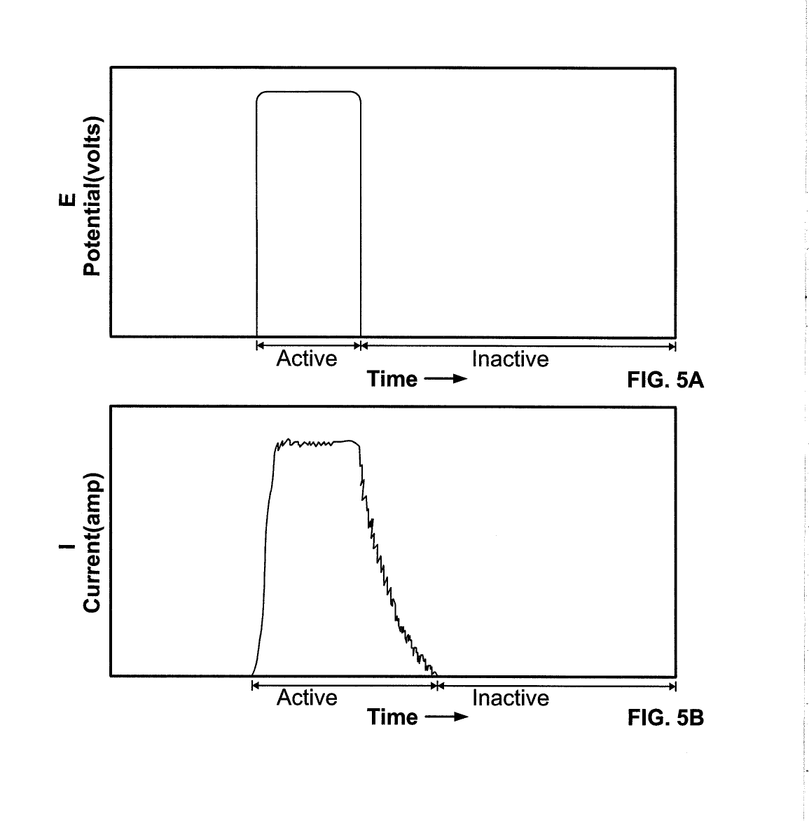

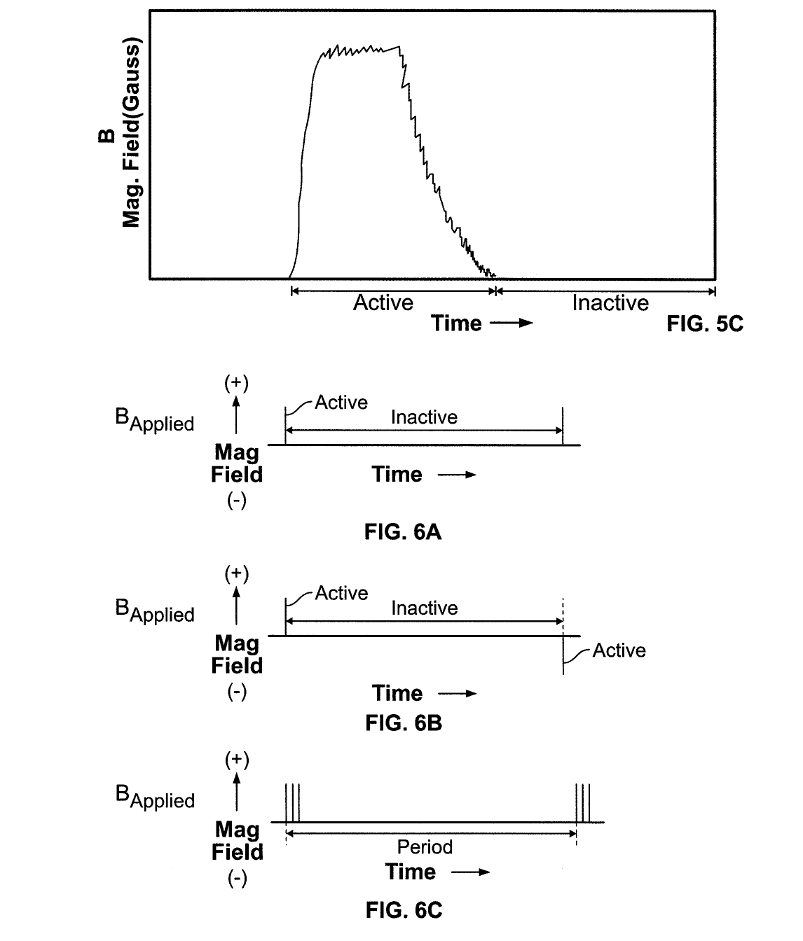

Referring now to FIGS. 5A, 5B, and 5C that depict a number of electromagnetic physical characteristics experienced by the magnetic field emitter 18 during active and inactive periods. FIG. 5A depicts a voltage step function across the magnetic field emitter 18 showing an almost instantaneous potential jump between two potential states (i.e., on state and off state). FIG. 5B depicts a current step function across the magnetic field emitter 18 showing an out of phase or delayed current, relative to the voltage step function, through the magnetic field emitter 18. FIG. 5C depicts a magnetic field step function emitted from the magnetic field emitter 18 showing an out of phase or delayed magnetic field, relative to the voltage step function, in which the magnetic field step function is approximately in phase with the current step function.

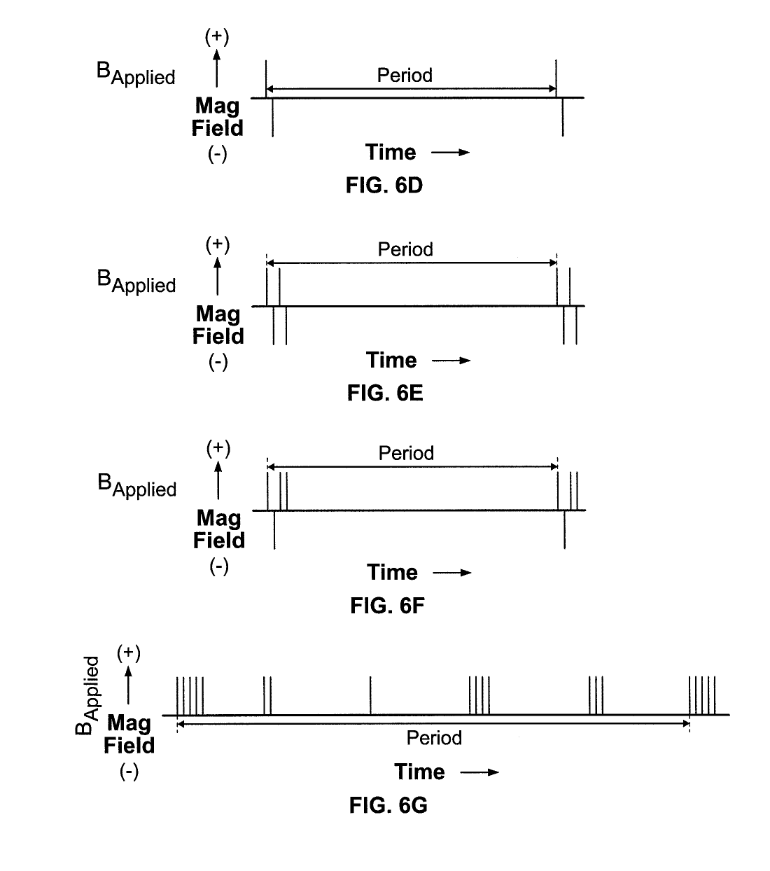

Referring now to FIGS. 6A, 6B, 6C, 6D, 6E, 6F, and 6G that depict a number of embodiments of showing different magnetic field output patterns as a function of time. The magnetic field emitter 18 is envisioned to be capable of producing any number of different patterns or modes of the resultant magnetic field along with being capable of producing alternating directional magnetic fields. As shown in FIG. 6A, one embodiment of the osteo or tissue healing device 10 provides that the magnetic field emitter 18 driven by the controlling circuit 14 can be configured to produce a unidirectional magnetic field for a short time period (i.e., during the active mode) and afterwards remain quiescent (i.e., the inactive mode) until the end of the cycle period. Quiescent conditions may be obtained by introducing a high impedance between the energizing circuit and the magnetic field generator, by grounding the electrical connectors of the energizing circuit, or by an intermediate state between such conditions. As this then affects and couples to the field and the target tissue activity, it is also considered an adjustable parameter or feature of the invention and one which may be optimized to obtain the desired target tissue regulation, effect, or response such as bone healing. As depicted in FIG. 6B another embodiment of the osteo or tissue healing device 10 provides that the magnetic field emitter 18 driven by the controlling circuit 14 can be configured to produce alternately magnetic field pulses in opposite directions, or polarity. Accordingly, the osteo or tissue healing device 10 is envisioned to be capable of producing the various magnetic field pulse patterns as depicted in FIGS. 6A, 6B, 6C, 6D, 6E, 6F, and 6G. that are illustrative and not limited to the infinite number of magnetic field pulse patterns that the osteo or tissue healing device 10 is envisioned to be capable of producing.

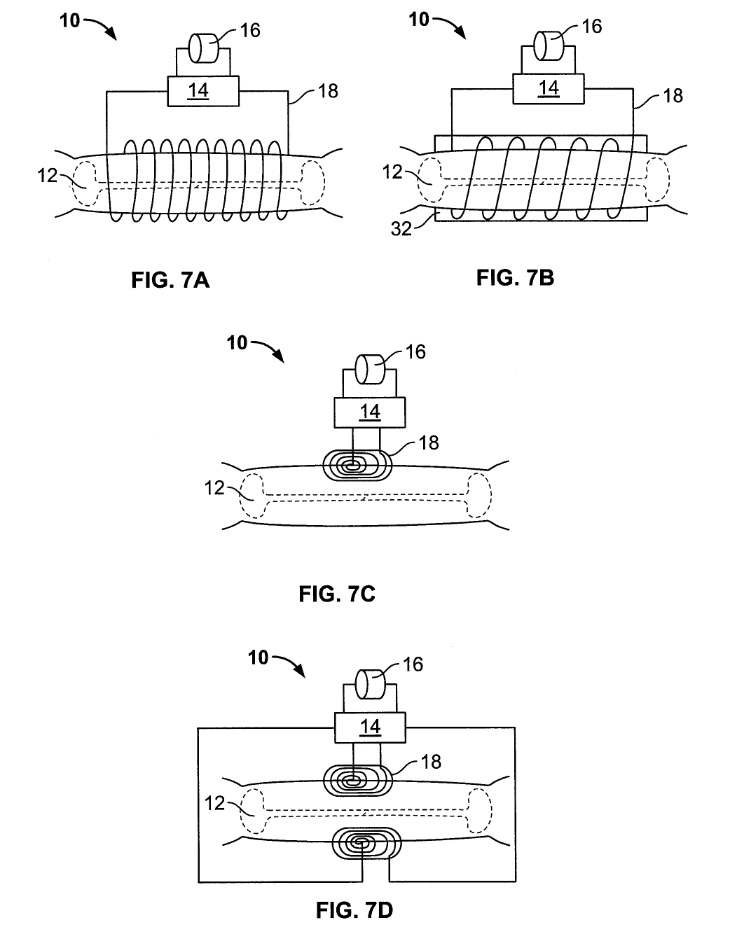

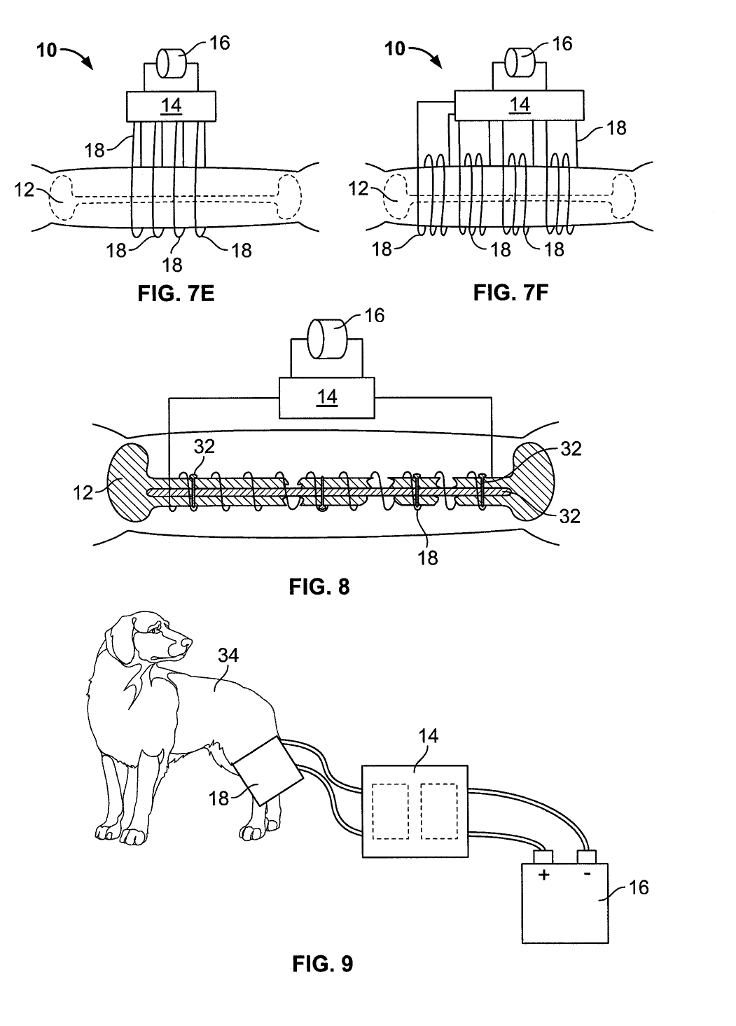

Referring now to FIGS. 7A, 7B, 7C, 7D, 7E, and 7F that depict various ways the osteo or tissue healing device 10 can be externally mounted to promote healing of a compromised bone 12 of a living mammal 34. Each figure (i.e., FIGS. 7A, 7B, 7C, 7D, 7E, and 7F) shows the controlling circuit 14 operationally coupled to the optional power source 16 and operationally coupled to at least one magnetic field emitter 18. FIG. 7B depicts that the magnetic field emitter 18 can be mounted within a stabilizing agent 32, such as a plaster of Paris cast.

Referring now to FIG. 8 that depicts the osteo or tissue healing device 10 internally mounted to promote healing of the compromised bone 12. FIG. 8 shows the controlling circuit 14 operationally coupled to the optional power source 16 and operationally coupled to a coil magnetic field emitter 18. Also shown in FIG. 8 is the stabilizing agent 32 depicted as a shank stabilizing agent 32 secured to the compromised bone 12 with screw stabilizing agents 32.

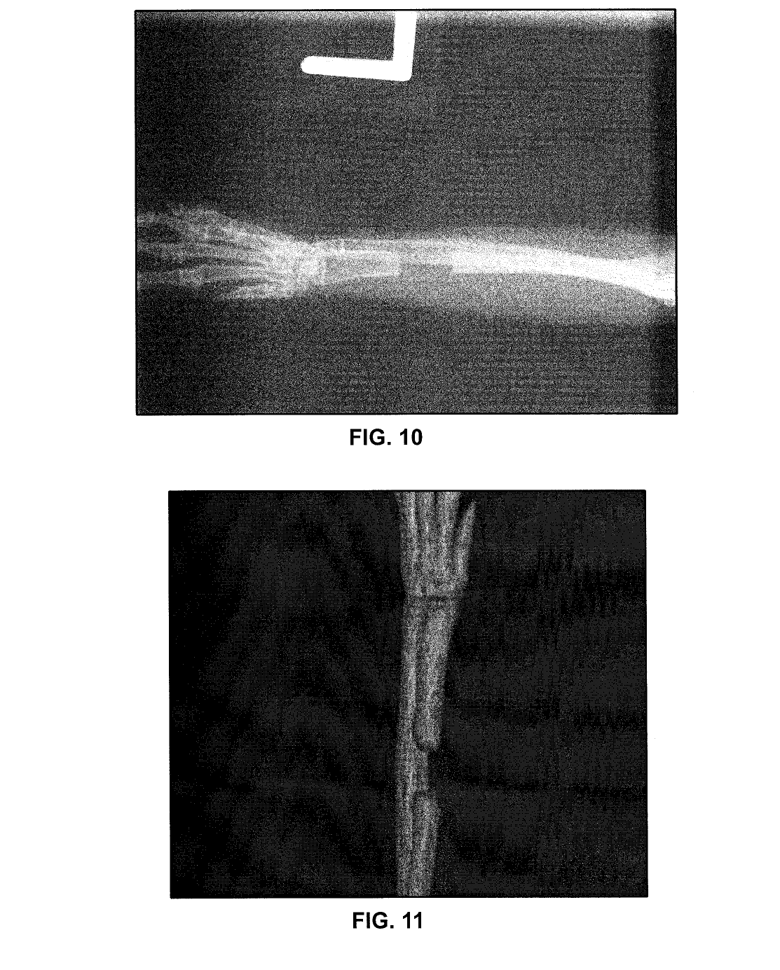

Referring now to FIG. 9 that depicts the osteo or tissue healing device 10 externally mounted onto a mammal 34. The osteo or tissue healing device 10 is shown composed of the magnetic field emitter 18 coupled to the controlling circuit 14 powered by the optional power source 16.

A study of bone healing was undertaken to test the effects of the present invention on the healing of the radial bone in New Zealand White Rabbits. In the tests, a piece of bone, approximately 2 cm long is removed from the radial bone of the rabbit. The surgical area was then closed and a coil was placed over the surgical area and the leg is wrapped with bandage to insure that the ulna bone did not subsequently break. A splint or cast was found not to be necessary as long as the ulna bone was provided support by tightly bandaging the leg. The coil was then provided with current pulses sufficient to provide a time variant magnetic field in accordance with this invention.

The surgery was performed on a test rabbit by making an incision and using an electrical surgical saw to remove approximately 2 cm of the radial bone. The incision was then closed. FIG. 10 depicts an X-ray of the leg of a test rabbit immediately post surgery showing the piece of the radial bone removed with the ulna bone intact.

After the X-ray was taken, the rabbits were taken to a clean room. A group of rabbits were used with each having the same surgical procedure. Half of the rabbits were provided the benefit of this invention and half were not. The test was a blind study with individuals involved not having access to which animals had the benefit of this invention. A coil was placed over the surgical area of each rabbit. On half of the rabbits, the coil was energized to provide a time varying electromagnetic force according to this invention. On the other half of the rabbits, the coil was not energized and thus did not have the benefit of this invention. After the coil was placed on the rabbit leg, the leg was bandaged with stiff bandaging to prevent the rabbits from breaking the ulna bone as they moved around their cages.

After two weeks, computed tomography scan (CTS) composite images were obtained of the surgical area on each of the rabbits. FIG. 11 shows the bone healing of a test rabbit having the benefit of this invention. As can be readily seen, the bone has begun healing and has almost breached the surgical gap, indicating a high initial healing rate.

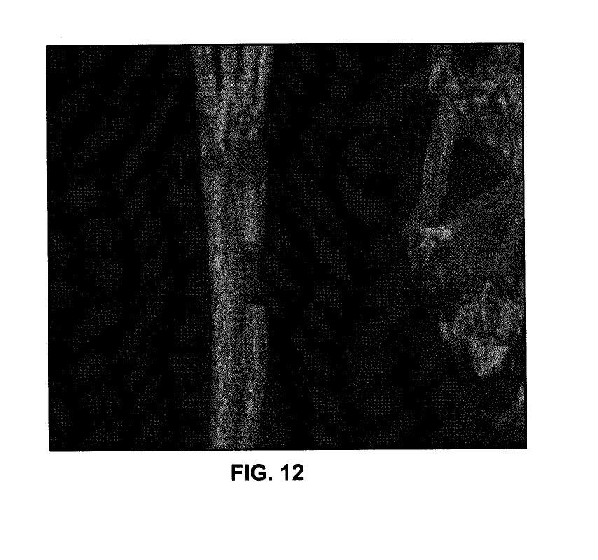

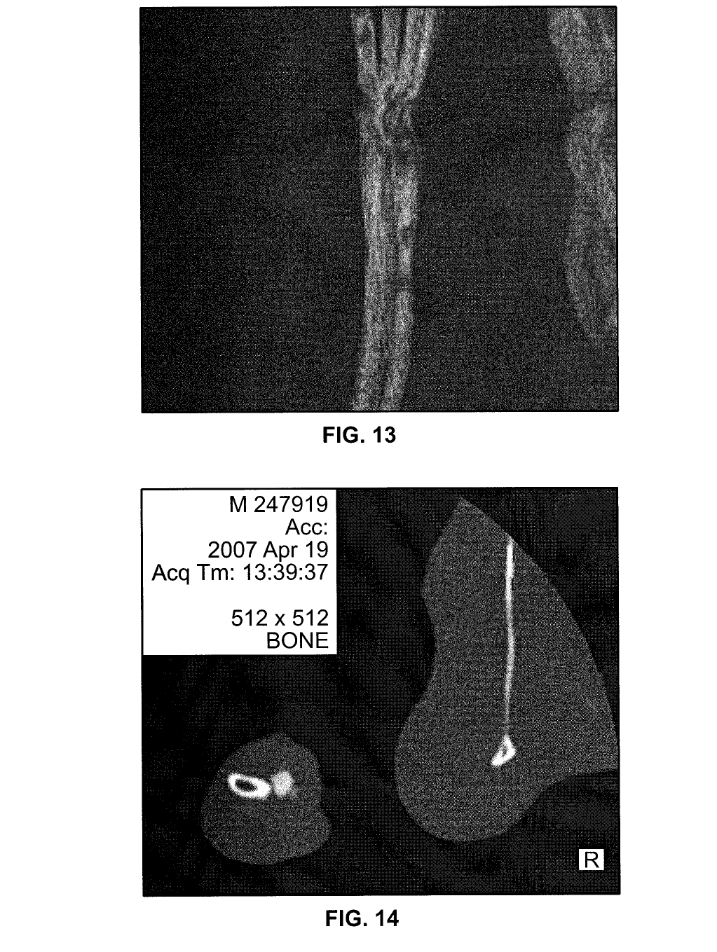

After four weeks, CTS scans were again taken of the surgical area on each of the rabbits. The scan in FIG. 12 depicts that the non-invention treated test rabbit again had no discernable healing of the bone after 4 weeks. FIG. 13, on the other hand, shows the bone healing of a test rabbit having the benefit of this invention after 4 weeks. The bone has completely healed and was deemed to be clinical healing by the surgeon.

FIG. 14 depicts a cross section of the surgical area of FIG. 14 showing that the bone has healed in a proper manner and that it is regenerated bone.

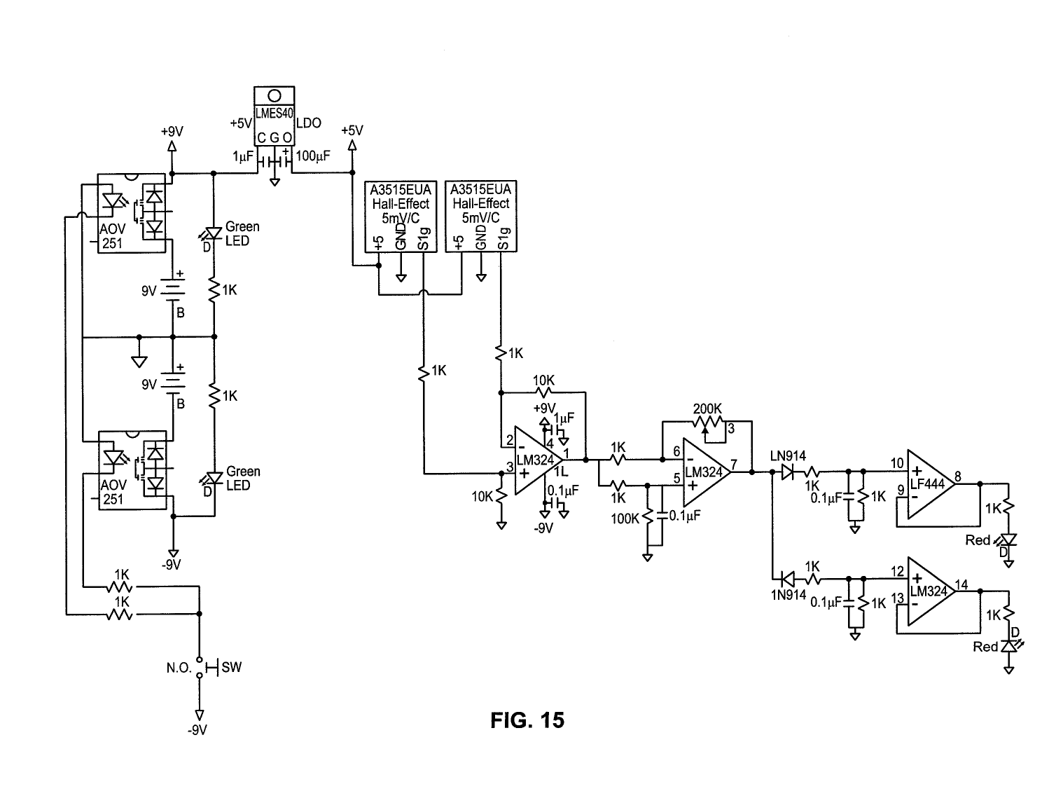

FIG. 15 depicts a schematic of the electronic circuit utilized to feed the time varying electromagnetic force to the coil.

As to the manner of usage and operation of the present invention, the same should be apparent from the above description. Accordingly, no further discussion relating to the manner of usage and operation will be provided.

While a number of embodiments of the osteo or tissue healing device has been described in detail, it should be apparent that modifications and variations thereto are possible, all of which fall within the true spirit and scope of the invention. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Throughout this specification, unless the context requires otherwise, the word “comprise” or variations such as “comprises” or “comprising” or the term “includes” or variations, thereof, or the term “having” or variations, thereof will be understood to imply the inclusion of a stated element or integer or group of elements or integers but not the exclusion of any other element or integer or group of elements or integers. In this regard, in construing the claim scope, an embodiment where one or more features is added to any of the claims is to be regarded as within the scope of the invention given that the essential features of the invention as claimed are included in such an embodiment.

Those skilled in the art will appreciate that the invention described herein is susceptible to variations and modifications other than those specifically described. It is to be understood that the invention includes all such variations and modifications that fall within its spirit and scope. The invention also includes all of the steps, features, compositions and compounds referred to or indicated in this specification, individually or collectively, and any and all combinations of any two or more of said steps or features.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

What is claimed is:

1. An osteo or tissue healing device for promoting healing of a compromised bone or tissue in a living mammal, said device comprising:
a magnetic field emitter electrically coupled to a controlling circuit, the magnetic field emitter configured to provide a time variant magnetic field when driven by an electric pulse train from the controlling circuit, the time variant magnetic field comprising a magnetic (B) field exhibiting a magnetic slew rate of at least about 10 kiloGauss/sec; and
the controlling circuit electrically coupled to the magnetic field emitter, wherein the controlling circuit configured to be powered by a power source, the controlling circuit configured to output the electric pulse train driving the magnetic field emitter.

2. The device of claim 1 wherein the B field is induced secondary to a time varying electric field.

3. The device of claim 1 wherein the B field is the magnetic field component of an emitted electromagnetic field.

4. The device of claim 1 wherein the time varying magnetic field is generated by current through a conductor.

5. The device of claim 2 wherein the time varying electric field is generated by a voltage across separated conductors.

6. The device of claim 3 wherein the electromagnetic field is emitted by an antenna carrying a time varying current

7. The device of claim 1 wherein the magnetic slew rate is a rising magnetic slew rate.

8. The device of claim 1 wherein the magnetic slew rate is a falling magnetic slew rate.

9. The device of claim 1 further comprising the power source electrically coupled to the controlling circuit.

10. The device of claim 9 wherein the power source is selected from the group consisting of a battery power source, a high capacity capacitor power source, and an electrical outlet power source.

11. The device of claim 1 wherein the magnetic field emitter is selected from the group consisting of a coil magnetic field emitter, a plurality of coil magnetic field emitters, a single loop magnetic field emitter, a plurality of loop magnetic field emitters, and an antenna magnetic field emitter.

12. The device of claim 1 wherein the magnetic field emitter has an inductance being at least 1 microHenry.

13. A kit for an osteo or tissue healing device for promoting healing of a compromised bone or tissue in a living mammal, said kit comprising:
a magnetic field emitter electrically coupleable to a controlling circuit, the magnetic field emitter configured to provide a time variant magnetic field when driven by an electric pulse train from the controlling circuit, the time variant magnetic field comprising a magnetic (B) field exhibiting a maximum slew rate of at least about 10 kiloGauss/sec; and
the controlling circuit electrically coupleable to the magnetic field emitter, wherein the controlling circuit configured to be powered by a power source, the controlling circuit configured to output the electric pulse train driving the magnetic field emitter.

14. The kit of claim 13 further comprising the power source configured to be electrically coupled to the controlling circuit.

15. The kit of claim 14 wherein the power source is selected from the group consisting of a battery, a high capacity capacitor, and an electrical outlet.

16. The kit of claim 13 further comprising a stabilizing agent 32.

17. The kit of claim 16 wherein the stabilizing agent is selected from the group consisting of an external applied plaster cast stabilizing agent, an externally applied splint stabilizing agent, an external traction mounting stabilizing agent and an internally applied shank stabilizing agent.

18. The kit of claim 13 wherein the magnetic field emitter is selected from the group consisting of a coil magnetic field emitter, a plurality of coil magnetic field emitters, a loop magnetic field emitter, a plurality of loop magnetic field emitters, and an antenna magnetic field emitter.

19. The kit of claim 13 wherein the magnetic field emitter has an inductance being at least about 1 microHenry.

20. A method for promoting healing of a compromised bone or tissue in a living mammal, said method comprising the step of applying a time variant magnetic field through the portion of the bone or tissue to promote healing of the bone or tissue, wherein the time variant magnetic field comprises a magnetic (B) field exhibiting a magnetic slew rate of at least about 10 kiloGauss/sec.

21. The method of claim 20 further comprising the step of aligning the bone or tissue in a desired orientation.

22. The method of claim 20 further comprising the step of stabilizing the bone or tissue with a stabilizing agent.

23. The method of claim 20 further comprising the step of mounting a magnetic field emitter near a portion of the bone or tissue.

24. The method of claim 20 wherein the compromised bone is selected from the group consisting of a simple fracture compromised bone, a compound fracture compromised bone, a cracked compromised bone, a strained compromised bone, and a low density compromised bone.

25. The method of claim 20 wherein the mammal is selected from the group consisting of a human, a domesticated dog, a domesticated cat, a rat, a mouse, a guinea pig, a rabbit, a horse, a cow, a llama, an alpaca, a mule, a donkey, a gorilla, a gibbon, an orangutan, a chimpanzee, a lemur, a rhinoceros, a monkey, a bat, a bison, a camel, a wolf, a coyote, a fox, a jackal, tiger, an oryx, a water buffalo, a elephant, a giraffe, an antelope, a deer, an elk, a lion, a cheetah, a panda, a leopard, a puma, a serval, an opossum, a kangaroo, a platypus, an armadillo, a lemur, a muskox, a baboon, a zebra, a pig, a koala, a tasmanian devil, a manatee, and a wombat.
